(19) United States
(12) Patent Application Publication
(10) Pub. No.: US 2024/0266051 A1
(43) Pub. Date: Aug. 8, 2024

(54) CARDIAC DATA ANALYSIS DEVICE, CARDIAC DATA ANALYSIS METHOD AND NON-TRANSITORY RECORDING MEDIUM

(71) Applicant: TOYOTA JIDOSHA KABUSHIKI KAISHA, Aichi-ken (JP)

(72) Inventor: Ippei FUKUTOMI, Machida-shi (JP)

(21) Appl. No.: 18/533,183

(22) Filed: Dec. 8, 2023

(30) Foreign Application Priority Data
Feb. 7, 2023 (JP) 2023-016937

(51) Int. Cl. G16H 50/20; A61B 5/347; A61B 5/352; A61B 5/364

(52) U.S. Cl. CPC G16H 50/20; A61B 5/347; A61B 5/352; A61B 5/364; G16H 40/67

(57) ABSTRACT

The cardiac data analysis device includes a processor configured to acquire cardiac data representing an electrocardiographic waveform or a cardiac acoustic waveform of a subject, frequency analyze the cardiac data to acquire analysis data, input the analysis data to a learned first discriminator to extract normal data from the analysis data, detect an interval between peak values of the same or different types in a waveform of the cardiac data, and determine whether an abnormality has occurred in a heart of the subject by inputting the normal data and the interval between the peak values to a learned second discriminator.

### FIELD

The present disclosure relates to a cardiac data analysis device, a cardiac data analysis method, and a non-transitory recording medium.

### BACKGROUND

Conventionally, an electrocardiographic waveform or a cardiac acoustic waveform representing a behavior of a heart of a subject is used for diagnosis of a heart disease of the subject. For example, Patent Literature 1 describes a technique for detecting an abnormality in the heart based on an electrocardiographic waveform.

The abnormality determination device described in Document 1 performs learning by the self-encoder using the time-varying waveform data obtained from the inherent motion of the object, and performs persistent homology conversion for calculating a change in the number of concatenated components in accordance with a change in the thresholds in the value-direction with respect to the waveform data. Further, the abnormality determination device determines an abnormality of the heart by inputting the output of the self-encoder based on the waveform data and the output of the persistent homology transform to the learned learner.

### CITATIONS LIST

### Patent Literature

[PTL 1] Japanese Unexamined Patent Publication No. 2020-036633

### SUMMARY

### Technical Problem

However, in the above-described method, not only the calculation by the learning unit but also the calculation by the self-encoding unit and the persistent homology conversion are performed, and thus the amount of calculation becomes very large.

In view of the above problem, an object of the present disclosure is to accurately detect an abnormality in a heart of a subject while suppressing an amount of calculation.

### Solution to Problem

The summary of the present disclosure is as follows.
(1) A cardiac data analysis device comprising: a processor configured to: acquire cardiac data representing an electrocardiographic waveform or a cardiac acoustic waveform of a subject; frequency analyze the cardiac data to acquire analysis data; input the analysis data to a learned first discriminator to extract normal data from the analysis data; detect an interval between peak values of the same or different types in a waveform of the cardiac data; and determine whether an abnormality has occurred in a heart of the subject by inputting the normal data and the interval between the peak values to a learned second discriminator.
(2) The cardiac data analysis device described in above (1), wherein the second discriminator is a recurrent neural network, and the processor is configured to determine whether an abnormality has occurred in the heart of the subject by inputting, in a time order, the normal data and the interval between the peak values for each frame having a predetermined time length to the second discriminator.
(3) The cardiac data analysis device described in above (1) or (2), wherein the subject is a vehicle occupant.
(4) A cardiac data analysis method executed by a computer, comprising: acquiring cardiac data representing an electrocardiographic waveform or a cardiac acoustic waveform of a subject; frequency analyzing the cardiac data to acquire analysis data; inputting the analysis data to a learned first discriminator to extract normal data from the analysis data; detecting an interval of between peak values of the same or different types in a waveform of the cardiac data; and determine whether an abnormality has occurred in a heart of the subject by inputting the normal data and the interval between the peak values to a learned second discriminator.
(5) A non-transitory recording medium having recorded thereon a computer program, the computer program causing a computer to: acquire cardiac data representing an electrocardiographic waveform or a cardiac acoustic waveform of a subject; frequency analyze the cardiac data to acquire analysis data; input the analysis data to a learned first discriminator to extract normal data from the analysis data; detect an interval between peak values of the same or different types in a waveform of the cardiac data; and determine whether an abnormality has occurred in a heart of the subject by inputting the normal data and the interval between the peak values to a learned second discriminator.

According to the present disclosure, it is possible to accurately detect an abnormality in a heart of a subject while suppressing an amount of calculation.

### BRIEF DESCRIPTION OF DRAWINGS

FIG. 1 is a schematic configuration diagram of a cardiac data analysis system according to an embodiment of the present disclosure.

FIG. 2 is a functional block diagram of the processor of the cardiac data analysis device.

FIG. 3 illustrates an example of cardiac data representing an electrocardiographic waveform.

FIG. 4A is a diagram showing an example of the frequency spectrum of the electrocardiographic waveform.

FIG. 4B is a diagram showing an example of the frequency spectrum of the electrocardiogram waveform.

FIG. 5 is a flowchart of a control routine of the abnormality determination process.

### DESCRIPTION OF EMBODIMENTS

Hereinafter, embodiments of the present disclosure will be described in detail with reference to the drawings. In the following description, the same reference numerals are given to the same constituent elements.

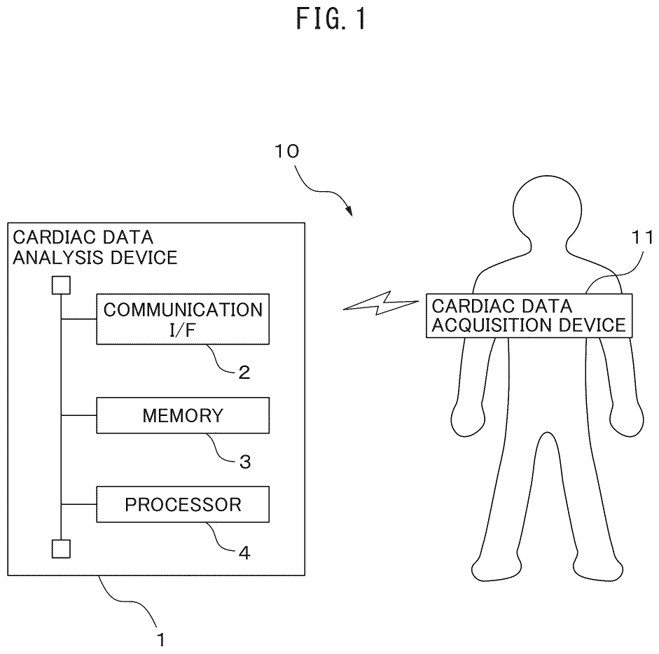

FIG. 1 is a schematic configuration diagram of a cardiac data analysis system 10 according to an embodiment of the present disclosure. The cardiac data analysis system 10 comprises a cardiac data acquisition device 11 and a cardiac data analysis device 1 for receiving cardiac data of a subject from the cardiac data acquisition device 11.

The cardiac data acquisition device 11 is attached to a subject and acquires cardiac data representing an electrocardiographic waveform or a cardiac acoustic waveform from the subject. For example, the cardiac data acquisition device 11 acquires cardiac data at predetermined intervals, transmits cardiac data to the cardiac data analysis device 1 each time the cardiac data is acquired, or transmits a predetermined number of cardiac data collectively to the cardiac data analysis device 1.

When the cardiac data acquiring device 11 acquires cardiac data representing an electrocardiographic waveform, the cardiac data acquiring device 11 is configured as an electrocardiograph, for example. On the other hand, when the cardiac data acquiring device 11 acquires cardiac data representing a cardiac acoustic waveform, the cardiac data acquiring device 11 is configured as, for example, a stethoscope. Note that the cardiac data acquiring device 11 may be configured as a device in which functions of an electrocardiograph and a stethoscope are integrated, or may include an electrocardiograph and a stethoscope, in order to acquire both cardiac data representing an electrocardiographic waveform and cardiac data representing a cardiac acoustic waveform.

The cardiac data analysis device 1 analyses the cardiac data acquired by the cardiac data acquiring device 11 and diagnoses an abnormal heart of the subject. As shown in FIG. 1, the cardiac data analysis device 1 comprises a communication interface 2, a memory 3 and a processor 4. The communication interface 2 and the memory 3 are connected to the processor 4 via signal lines. The cardiac data analysis device 1 may further include a user interface such as a touch panel, a keyboard, or a display. The cardiac data analysis device 1 may further include a storage device including a hard disk drive (HDD), a solid state drive (SSD), or an optical recording medium and an accessing device thereof.

The communication interface 2 has an interface circuitry for connecting the cardiac data analysis device 1 and the outside of the cardiac data analysis device 1 (for example, the cardiac data acquiring device 11) via a wired or wireless predetermined communication line. The cardiac data analysis device 1 communicates with the cardiac data acquiring device 11 via the communication interface 2. The communication interface 2 is an example of a communication unit of the cardiac data analysis device 1.

The memory 3 includes, for example, a volatile semiconductor memory and a non-volatile semiconductor memory. The memory 3 stores programs and data used when various kinds of processing are executed by the processor 4, data generated by the processor 4, and the like. The memory 3 is an example of a storage unit of the cardiac data analysis device 1.

The processor 4 comprises one or more CPU (Central Processing Unit) and its peripheral circuitry. The processor 43 may further include an arithmetic circuit such as a logical operation unit, a numerical operation unit, or a graphics processing unit.

In the present embodiment, the subject to be diagnosed with abnormality of the heart is an occupant of the vehicle. That is, the cardiac data acquiring device 11 is worn on a subject in a vehicle and used in the vehicle. Thus, the cardiac data analysis device 1 can diagnose an abnormal heart of a subject when the subject is riding on a vehicle. Examples of vehicles include vehicles such as automobiles, motorcycles or trains, aircraft, spacecraft, and the like.

The cardiac data analysis device 1 is mounted on a vehicle. For example, if the subject is an occupant of an automobile, an electronic control unit (ECU) mounted on the automobile functions as the cardiac data analysis device 1. Note that a mobile terminal such as a smart phone of a subject or a server provided outside the vehicle may function as the cardiac data analysis device 1. When the server is used as the cardiac data analysis device 1, the cardiac data analysis device 1 communicates with the cardiac data acquiring device 11 via a communication network, such as an internet network, using a communication module or the like provided in the vehicle. The cardiac data analysis device 1 may be a dedicated terminal for analyzing cardiac data.

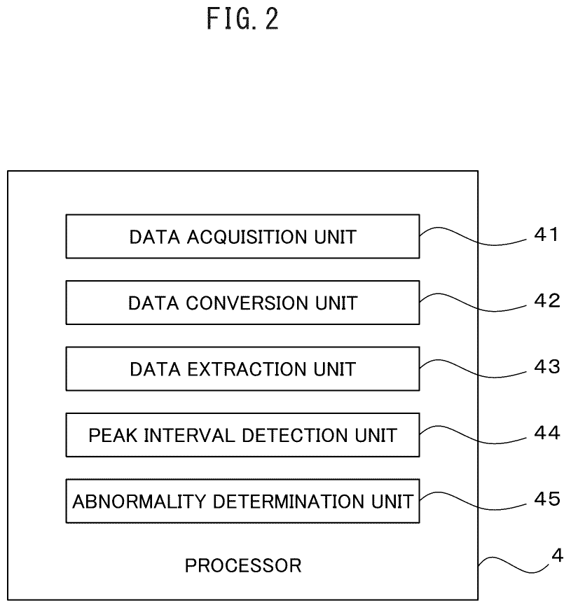

FIG. 2 is a functional diagram of the processor 4 of the cardiac data analysis device 1. In the present embodiment, the processor 4 includes a data acquisition unit 41, a data conversion unit 42, a data extraction unit 43, a peak interval detection unit 44, and an abnormality determination unit 45. The data acquisition unit 41, the data conversion unit 42, the data extraction unit 43, the peak interval detection unit 44, and the abnormality determination unit 45 are functional modules realized by the processor 4 executing a computer program stored in the memory 3. Note that these functional modules may be realized by a dedicated arithmetic circuit provided in the processor 4.

The data acquisition unit 41 acquires cardiac data of the subject. Specifically, the data acquisition unit 41 acquires cardiac data by receiving cardiac data from the cardiac data acquisition device 11 via the communication interface 2.

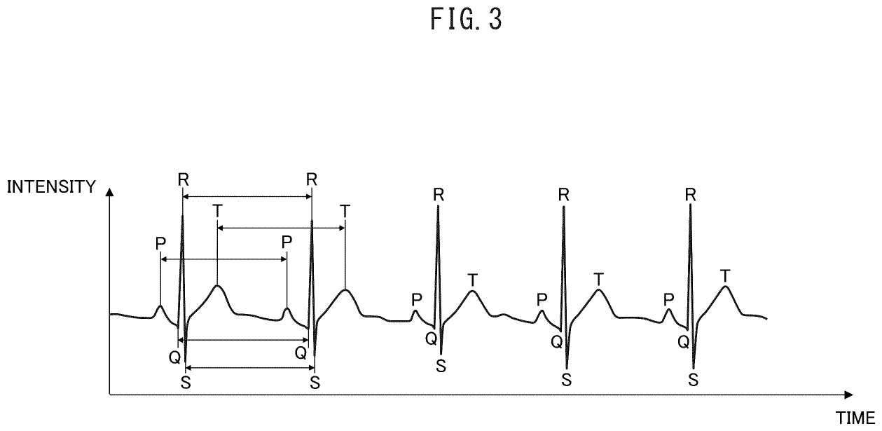

FIG. 3 is a diagram showing an illustration of cardiac datum representing an electrocardiographic waveform. In FIG. 3, the abscissa represents time, and the ordinate represents the intensity of the electrocardiographic signal. The electrocardiographic waveform is mainly composed of three waveforms: a P wave, a QRS wave, and a T wave. These three major waveforms appear every single beat of the heart. The P wave is a waveform caused by depolarization of the atrium (excitation of the atrium), and has a peak value (P peak value) at which the electrocardiographic signal is maximized. The QRS wave is a waveform caused by depolarization of the ventricle (excitation of the ventricle), and is composed of three waveforms: a Q wave, an R wave, and an S wave. The Q wave is a downward waveform appearing after the P wave, and has a peak value (Q peak value) at which the electrocardiographic signal becomes minimum. The R wave is an upward waveform appearing after the Q wave, and has a peak value (R peak value) at which the electrocardiographic signal becomes maximized. The S wave is a downward waveform appearing after the R wave, and has a peak value (S peak value) at which the electrocardiographic signal becomes minimum. The T wave is a waveform caused by repolarization of the ventricle (accommodation of ventricular excitation), and has a peak value (T peak value) at which the electrocardiographic signal becomes maximized.

The data conversion unit 42 converts the cardiac data acquired by the data acquisition unit 41 into analysis data. Specifically, the data conversion unit 42 performs frequency analysis on the cardiac data acquired by the data acquisition unit 41 to acquire analysis data. At this time, the data conversion unit 42 divides the cardiac data in units of frames having a predetermined time length, and performs frequency analysis on each of the frames of the cardiac data. The time length of the frame is preferably set to a length of about several seconds to several tens of seconds so that a single frame includes a plurality of peak values of a predetermined type of waveform (for example, an R wave).

Examples of a frequency-analysis technique used for converting cardiac data into analysis data include a Fast Fourier Transform (FFT), a Short-Time Fourier Transform (STFT), and a Wavelet Transform. When the frequency analysis by the fast Fourier transform is performed, the data conversion unit 42 transforms the heart data in the time domain into a frequency spectrum, and acquires the frequency spectrum as analysis data. On the other hand, when the frequency analysis by the short-time Fourier transform or the wavelet transform is performed, the data conversion unit 42 time-frequency-transforms the heart data in the time domain and acquires the time-frequency-transformed data as the analysis data. When the cardiac data representing the electrocardiographic waveform is acquired by the data acquisition unit 41, the cardiac data is preferably converted into analysis data using a fast Fourier transform. On the other hand, in a case where the cardiac data representing the cardiac acoustic waveform is acquired by the data acquisition unit 41, the cardiac data is preferably converted into analysis data using wavelet transform.

In the analysis data in which the frequency analysis is performed, the characteristics of the waveform are emphasized in comparison with the original heart data. Therefore, it is possible to improve the accuracy of the abnormality diagnosis by using the analysis data for diagnosing the abnormality of the heart of the subject. However, when noise is included in the original cardiac data, the noise is also superimposed on the analysis data, and the accuracy of the abnormality diagnosis is deteriorated. Noise is caused by movement of the subject's muscles, vibrate or sound of the vehicle, etc. Therefore, when the subject is an occupant of the vehicle, the influence of noise becomes particularly remarkable.

Therefore, the data extraction unit 43 extracts normal data with less noise from the analysis data acquired by the data conversion unit 42, and removes noise data with more noise. Specifically, the data extraction unit 43 inputs the analysis data acquired by the data conversion unit 42 to the learned first discriminator to extract normal data from the analysis data. In other words, the data extraction unit 43 inputs the analysis data to the learned first classifier to remove the noise data from the analysis data. Thus, the influence of noise on the abnormality diagnosis can be reduced.

The first classifier is learned in advance so as to output an identification result from the analysis data, that is, learned in advance so as to classify the analysis data as either normal data or noise data. For example, the data extraction unit 43 uses a multi-layer perceptron neural network as the first discriminator. In this case, the first classifier includes an input layer to which the analysis data is input, an output layer to which the identification result is output, and one or more hidden layers between the input layer and the output layer. The number of layers of the hidden layer is preferably set to 2 or more, and the number of nodes of each hidden layer is preferably set to 8 or more or 12 or more. The learning of the first classifier is performed using a large number of pieces of teacher data by a supervised learning method such as an error back propagation method, for example. As the first classifier, a machine learning model other than a neural network, for example, a machine learning model such as a support vector machine or a random forest may be used.

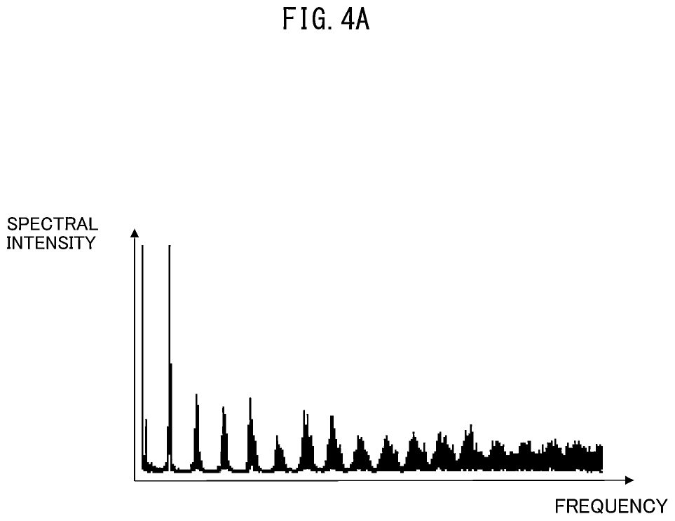

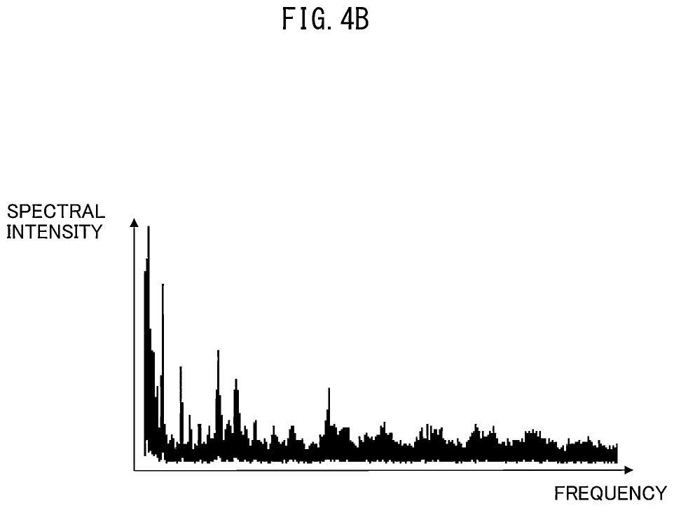

Each of FIG. 4 A and FIG. 4 B is a diagram showing an example of the frequency spectrum of the electrocardiographic waveform. In FIG. 4 A and FIG. 4 B, the horizontal axis represents frequency, and the vertical axis represents spectral intensity. For example, when the analysis data of the frequency spectrum shown in FIG. 4 A is input to the first discriminator, the normal data is output as the identification result, and when the analysis data of the frequency spectrum shown in FIG. 4 B is input to the first discriminator, the noise data is output as the identification result. Consequently, the data extraction unit 43 extracts the frequency spectrum indicated by FIG. 4 A as normal data, and removes the frequency spectrum indicated by FIG. 4 B as noise data.

The peak interval detection unit 44 detects the interval between peak values of the same or different types in the waveform of the cardiac data. When cardiac data representing an electrocardiographic waveform is used for abnormality diagnosis, the peak interval detection unit 44 detects the interval between peak values of the same or different types in the electrocardiographic waveform. As shown in FIG. 3, the electrocardiographic waveform has P-peak value, Q-peak value, R-peak value, S-peak value and T-peak value as peak values.

First, the peak interval detection unit 44 divides the cardiac data in units of frames in the same manner as the data conversion unit 42, and detects the peak value in the waveform of the cardiac data with respect to the cardiac data of the same frame as the normal data extracted by the data extraction unit 43. For example, the peak interval detection unit 44 detects the peak value in the waveform of the cardiac data by detecting the maximum value and the minimum value of the electrocardiographic signal. Then, the peak interval detection unit 44 calculates a difference between the appearance timings of the two peak values to calculate an interval between these peak values.

For example, the peak interval detection unit 44 detects at least two intervals, preferably three or more intervals, among the intervals between two consecutive peak values of the same type in the electrocardiographic waveform. FIG. 3 shows the interval between two consecutive P-peak values and the interval between two consecutive Q-peak values, the interval between two consecutive R-peak values, and the interval between two consecutive S-peak values and the interval between two consecutive T-peak values.

Note that the peak interval detection unit 44 may detect at least two intervals, preferably three or more intervals, among the intervals between two consecutive peak values of different types in the electrocardiographic waveform. Examples of the interval between two consecutive peak values of different types are the interval between the P peak value and the next Q peak value, the interval between the Q peak value and the next R peak value, the interval between the R peak value and the next S peak value, the interval between the S peak value and the next T peak value, and the interval between the T peak value and the next P peak value. Further, the peak interval detection unit 44 may detect at least two intervals, preferably three or more intervals, among the intervals between two consecutive peak values of the same type in the electrocardiographic waveform and the intervals between two consecutive peak values of different types in the electrocardiographic waveform.

On the other hand, when cardiac data representing a cardiac acoustic waveform is used for abnormality diagnosis, the peak interval detection unit 44 detects intervals between peak values of the same or different types in the cardiac acoustic waveform. The cardiac acoustic waveform is mainly composed of waveforms of a first sound and a waveform of a second sound. The first sound is a sound when the mitral valve and the tricuspid valve are closed, and the waveform has a peak value (first sound peak value) at which the cardiac acoustic signal is maximized. The second sound is a sound when the aortic valve and the pulmonary valve are closed, and the waveform has a peak value (second sound peak value) at which the cardiac acoustic signal is maximized. For example, the peak interval detection unit 44 detects the peak value in the waveform of the cardiac data by detecting the maximum value of the cardiac acoustic signal. Then, the peak interval detection unit 44 calculates a difference between the appearance timings of the two peak values to calculate an interval between these peak values.

For example, the peak interval detection unit 44 detects at least two intervals among the intervals between two consecutive peak values of the same type in the cardiac acoustic waveform. Examples of the interval between two consecutive peak values of the same type are the interval between two consecutive first sound peak values and the interval between two consecutive second sound peak values.

Note that the peak interval detection unit 44 may detect at least two intervals among the intervals of two consecutive peak values of different types in the cardiac acoustic waveform. Examples of the interval between two consecutive peak values of different types are the interval between the first sound peak value and the subsequent second sound peak value, and the interval between the second sound peak value and the subsequent first sound peak value. In addition, the peak interval detection unit 44 may detect at least two intervals, preferably three or more intervals, among the intervals of two consecutive peak values of the same type in the cardiac acoustic waveform and the intervals of two consecutive peak values of different types in the cardiac acoustic waveform.

In the present embodiment, in order to diagnose the abnormality of the heart of the subject, the interval between the peak values detected by the peak interval detection unit 44 is used in addition to the normal data extracted by the data extraction unit 43. As a result, the characteristic quantity of the heart used for abnormality diagnosis can be increased, and thus the accuracy of abnormality diagnosis can be improved.

Specifically, the abnormality determination unit 45 determines whether an abnormality has occurred in the heart of the subject by inputting the normal data and the interval between the peak values to the learned second discriminator. The second classifier is learned in advance to output the presence or absence of an abnormality in the heart from the normal data and the interval between the peak values. For example, the abnormality determination unit 45 uses a multi-layer perceptron neural network as the second discriminator. In this case, the second discriminator includes an input layer to which the normal data and the interval between the peak values are input, an output layer to which the determination result is output, and one or more hidden layers between the input layer and the output layer. The number of layers of the hidden layer is preferably set to 2 or more, and the number of nodes of each hidden layer is preferably set to 8 or more or 12 or more. The learning of the second classifier is performed using a large number of pieces of teacher data by a supervised learning method such as an error back propagation method, for example. As the second classifier, a machine learning model other than a neural network, for example, a machine learning model such as a support vector machine or a random forest may be used.

The abnormality determination unit 45 inputs the normal data and the interval between the peak values in the same frame as the normal data to the second discriminator. Then, the abnormality determination unit 45 determines that an abnormality has occurred in the heart of the subject when the output result of the second discriminator is abnormal, and determines that an abnormality has not occurred in the heart of the subject when the output result of the second discriminator is abnormal.

Note that the abnormality determination unit 45 may determine whether an abnormality has occurred in the heart of the subject by inputting each of a plurality of combinations of the normal data and the intervals between peak values in the same frame to the second discriminator. In this case, the abnormality determination unit 45 may determine that an abnormality has occurred in the heart of the subject only when the output results of the second discriminator for frames of a predetermined number (for example, 2) or a predetermined ratio or more are abnormal.

In the abnormality diagnosis method described above, by using the first discriminator and the second discriminator, it is possible to efficiently detect the characteristics of the cardiac data, and it is possible to accurately detect the abnormality of the heart of the subject while suppressing the calculation amount.

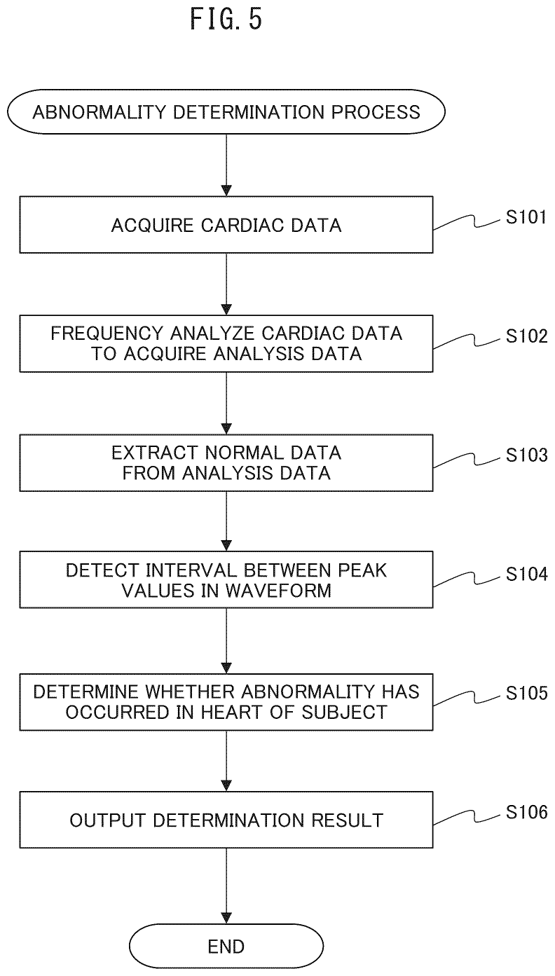

Hereinafter, the above-described control process flow will be described by referring to FIG. 5. FIG. 5 is a flow chart showing the control routine of the abnormality determination process. The control routine is repeatedly executed by the processor 4 (the data acquisition unit 41, the data conversion unit 42, the data extraction unit 43, the peak interval detection unit 44, and the abnormality determination unit 45) of the cardiac data analysis device 1 at predetermined execution intervals.

Initially, in the step S101, the data acquisition unit 41 acquires cardiac data by receiving cardiac data from the cardiac data acquisition device 11.

Next, in the step S102, the data conversion unit 42 divides the cardiac data in units of frames having a predetermined time length, performs frequency analysis on each of the frames of the cardiac data, and acquires a plurality of pieces of analysis data for each frame.

Next, in the step S103, the data extraction unit 43 inputs the analysis data acquired by the data conversion unit 42 to the learned first discriminator, and extracts normal data from the analysis data. That is, the data extraction unit 43 extracts the analysis data identified as normal data by the first classifier among the plurality of analysis data acquired by the data conversion unit 42, and removes the analysis data identified as noise data by the first classifier.

Next, in the step S104, the peak interval detection unit 44 detects a predetermined type of peak values in the waveform of the cardiac data for each of the cardiac data of the same frame as the normal data extracted by the data extracting unit 43, and detects an interval between the predetermined type of peak values.

Next, in the step S105, the abnormality determination unit 45 determines whether an abnormality has occurred in the heart of the subject by inputting the normal data extracted by the data extraction unit 43 and the interval between the peak values detected by the peak interval detection unit 44 to the learned second discriminator. When the output result of the second discriminator is abnormal, the abnormality determination unit 45 determines that an abnormality has occurred in the heart of the subject. On the other hand, when the output result of the second discriminator is no abnormality, the abnormality determination unit 45 determines that no abnormality has occurred in the heart of the subject.

Next, in the step S106, the abnormality determination unit 45 outputs the determination result of the abnormality diagnosis to the subject via the user interface of the cardiac data analysis device 1 or another device (for example, a user interface provided in the vehicle or a mobile terminal of the subject). As a result, the subject can easily know the determination result of the abnormality diagnosis. Note that the second discriminator may be configured to output the type of abnormality of the heart, and the abnormality determination unit 45 may output the determination result including not only the presence or absence of the abnormality but also the type of the abnormality. After the step S106, the control routine ends.

The control routine in FIG. 5, the order of the steps S102 and S103 and the step S104 may be changed. Further, the process of step S102 and S103 and the process of step S104 may be executed in parallel.

While preferred embodiments of the present disclosure have been described above, the present disclosure is not limited to these embodiments, and various modifications and changes can be made within the scope of the claims. For example, both cardiac data representing an electrocardiographic waveform and cardiac data representing a cardiac acoustic waveform may be used to diagnose an abnormality in a subject's heart.

In addition, the peak interval detection unit 44 may detect a peak value in the waveform of the cardiac data by applying the cardiac data to the bandpass filter in order to detect the interval of the peak values. As a result, the influence of noise included in the cardiac data can be reduced, and the detection accuracy of the interval between the peak values can be improved. The bandpass filter attenuates frequency components other than the predetermined frequency band, and the predetermined frequency band is set to, for example, 1 to 100 Hz, preferably 2 to 40 Hz. In addition, in a case where a plurality of intervals of peak values calculated as a difference between two specific peak values (for example, two consecutive P peak values) are included in one frame, the peak interval detection unit 44 may detect the interval of the peak values as an average value of the intervals of the plurality of peak values.

The second discriminator used by the abnormality determination unit 45 may be a recurrent neural network (RNN: Recurrent Neural Network). In this case, the abnormality determination unit 45 determines whether an abnormality has occurred in the heart of the subject by inputting the normal data and the interval between the peak values for each frame having a predetermined time length to the second discriminator in time order. As a result, it is possible to determine the presence or absence of an abnormality in the heart in consideration of a time-series change in the heart data, and thus it is possible to improve the accuracy of the abnormality diagnosis. Note that the second classifier configured as a RNN may have a gated RNN layer such as LSTM or GRU as one of the hidden layers.

Further, the abnormality determination unit 45 may perform weighting on the input values as preprocessing for the input values (the normal data and the interval between peak values) input to the second discriminator. For example, the abnormality determination unit 45 increases the weight coefficient for each value of the normal data as the confidence level output by the first classifier when the analysis data is classified as the normal data increases. As another example, the abnormality determination unit 45 increases the weight coefficient with respect to the interval between the peak values as the reliability of the peak values increases. In this case, for example, the abnormality determination unit 45 determines that the reliability of the peak value is higher as the difference between the peak value and the value of the waveform before and after the peak value is larger.

In addition, a computer program for causing a computer to realize the functions of the respective units included in the processor 4 of the cardiac data analysis device 1 may be provided in a form stored in a computer-readable recording medium. The computer-readable recording medium is, for example, a magnetic recording medium, an optical recording medium, or a semiconductor memory.

### REFERENCE SIGNS LIST

1 Cardiac data analysis device
4 Processor
41 Data acquisition unit
42 Data conversion unit
43 Data extraction unit
44 Peak interval detection unit
45 Abnormality determination unit

1. A cardiac data analysis device comprising a processor configured to:
acquire cardiac data representing an electrocardiographic waveform or a cardiac acoustic waveform of a subject;
frequency analyze the cardiac data to acquire analysis data;
input the analysis data to a learned first discriminator to extract normal data from the analysis data;
detect an interval between peak values of the same or different types in a waveform of the cardiac data; and
determine whether an abnormality has occurred in a heart of the subject by inputting the normal data and the interval between the peak values to a learned second discriminator.

2. The cardiac data analysis device according to claim 1, wherein
the second discriminator is a recurrent neural network, and
the processor is configured to determine whether an abnormality has occurred in the heart of the subject by inputting, in a time order, the normal data and the interval between the peak values for each frame having a predetermined time length to the second discriminator.

3. The cardiac data analysis device according to claim 1, wherein the subject is a vehicle occupant.

4. A cardiac data analysis method executed by a computer, comprising:
acquiring cardiac data representing an electrocardiographic waveform or a cardiac acoustic waveform of a subject;
frequency analyzing the cardiac data to acquire analysis data;
inputting the analysis data to a learned first discriminator to extract normal data from the analysis data;
detecting an interval of between peak values of the same or different types in a waveform of the cardiac data; and
determine whether an abnormality has occurred in a heart of the subject by inputting the normal data and the interval between the peak values to a learned second discriminator.

5. A non-transitory recording medium having recorded thereon a computer program, the computer program causing a computer to:
acquire cardiac data representing an electrocardiographic waveform or a cardiac acoustic waveform of a subject;
frequency analyze the cardiac data to acquire analysis data;
input the analysis data to a learned first discriminator to extract normal data from the analysis data;
detect an interval between peak values of the same or different types in a waveform of the cardiac data; and
determine whether an abnormality has occurred in a heart of the subject by inputting the normal data and the interval between the peak values to a learned second discriminator.
